(19) United States
(12) Patent Application Publication
(10) Pub. No.: US 2023/0119563 A1
(43) Pub. Date: Apr. 20, 2023

(54) METHOD, SYSTEM, AND NON-TRANSITORY COMPUTER-READABLE RECORDING MEDIUM FOR PROVIDING INFORMATION ABOUT POST-CARDIAC ARREST PROGNOSIS

(71) Applicants: SEOUL NATIONAL UNIVERSITY HOSPITAL, Seoul (KR); KOREA ADVANCED INSTITUTE OF SCIENCE AND TECHNOLOGY, Daejeon (KR); OBE LAB., INC., Seoul (KR)

(72) Inventors: Sang Bae KO, Seoul (KR); Tae Jung KIM, Seoul (KR); Hyeon Min BAE, Daejeon (KR); Jong Kwan CHOI, Seoul (KR); Jae Myoung KIM, Daejeon (KR)

(73) Assignees: SEOUL NATIONAL UNIVERSITY HOSPITAL, Seoul (KR); KOREA ADVANCED INSTITUTE OF SCIENCE AND TECHNOLOGY, Daejeon (KR); OBE LAB., INC., Seoul (KR)

(21) Appl. No.: 17/909,204

(22) PCT Filed: Mar. 5, 2021
(86) PCT No.: PCT/KR2021/002784

§ 371 Date: Sep. 2, 2022

(30) Foreign Application Priority Data
Mar. 5, 2020 (KR) 10-2020-0027562

(51) Int. Cl. A61B 5/00; A61B 5/145

(52) U.S. Cl. CPC A61B 5/7275; A61B 5/14546; A61B 5/0075; A61B 5/726; A61B 5/7246; A61B 5/7264

(57) ABSTRACT

There is provided a method of providing information about prognosis after cardiac arrest. The method includes the steps of: calculating biological information based on a signal relating to a hemoglobin concentration measured from a cerebral region of a subject to be measured; and providing the information about the prognosis after cardiac arrest of the subject with reference to the calculated biological information and a biomarker relating to the prognosis after cardiac arrest measured from a blood of the subject.

### CROSS-REFERENCE TO RELATED APPLICATIONS

This application is a national phase of Patent Cooperation Treaty (PCT) International Application No. PCT/KR2021/002784 filed on Mar. 5, 2021, which claims priority to Korean Patent Application No. 10-2020-0027562 filed on Mar. 5, 2020. The entire contents of PCT International Application No. PCT/KR2021/002784 is hereby incorporated by reference.

### TECHNICAL FIELD

The present disclosure relates to a method, system, and non-transitory computer-readable recording medium for providing information about a prognosis after a cardiac arrest.

### BACKGROUND

Timely and accurately predicting the neurological prognosis of a patient resuscitated after cardiac arrest is very important in the treatment and management of the patient.

However, according to a previously-introduced prediction method, merely a neurologically poor prognosis can be predicted. In addition, in predicting the neurological prognosis, parameters, which may be affected by a sedative drug, a neuromuscular blocker or the like used when applying a targeted temperature management (TTM) to a patient resuscitated after cardiac arrest in an acute stage, are utilized. Thus, there is a limitation in that it is difficult to predict the prognosis accurately (and early) in the acute stage.

Near-infrared spectroscopy (NIRS) that has been introduced recently is a method of indirectly analyzing a bioactivity occurring in a body portion (e.g., brain or the like) of the person by measuring a degree of attenuation of near-infrared ray (due to scattering and absorption by oxidized or non-oxidized hemoglobin) which varies with a change in hemodynamic (e.g., concentrations of oxy hemoglobin and deoxy hemoglobin) which occurs in the body portion. As an example, a case of measuring hemodynamic changes occurring in the brain will be described in detail. Near-infrared ray having a wavelength range of about 630 nm to 1,300 nm may be transmitted through the skull of the person and reach the depth of about 1 cm to 3 cm from the skull. By irradiating such near-infrared ray to the head of the person and detecting the near-infrared ray reflected or scattered therefrom, it is possible to measure a change in hemodynamic (e.g., concentrations of oxygen in the blood (i.e., oxidized hemoglobin) or the like) occurring in the cerebral cortex of the person.

More specifically, according to the near-infrared spectroscopy, the neural activity occurring in the brain (particularly, cortex) of the person may be quantified by arranging at least one light irradiation unit (near-infrared ray irradiation module) and at least one light detection unit (near-infrared ray sensing module) at predetermined intervals in various sections of the head of the person, and analyzing signals relating to hemodynamics (e.g., optical density (OD) signals based on the near-infrared spectroscopy) obtained by the at least one light irradiation unit and the at least one light detection unit.

In view of this, the present inventors proposed a novel and advanced technique capable of providing information about prognosis after cardiac arrest of a subject to be measured by referring to a signal relating to a hemoglobin concentration that may be measured with a near-infrared spectroscopy and a biomarker that may be measured from the blood of the subject.

### SUMMARY

An object of the present disclosure is to solve the above-described problems in the prior art.

Another object of the present disclosure is to provide information about prognosis after cardiac arrest of a subject to be measured by calculating biological information based on a signal relating to a hemoglobin concentration measured from a cerebral region of the subject, and referring to the calculated biological information and a biomarker relating to the prognosis after cardiac arrest measured from a blood of the subject.

Representative configurations of the present disclosure to achieve the above objects are described below.

According to one aspect of the present disclosure, there is provided a method of providing information about prognosis after cardiac arrest, the method including the steps of: calculating biological information based on a signal relating to a hemoglobin concentration measured from a cerebral region of a subject to be measured; and providing the information about the prognosis after cardiac arrest of the subject with reference to the calculated biological information and a biomarker relating to the prognosis after cardiac arrest measured from a blood of the subject.

According to another aspect of the present disclosure, there is provided a system for providing information about prognosis after cardiac arrest, the system including: a biological information management unit configured to calculate biological information based on a signal relating to a hemoglobin concentration measured from a cerebral region of a subject to be measured; and a prognosis information provision unit configured to provide the information about the prognosis after cardiac arrest of the subject with reference to the calculated biological information and a biomarker relating to the prognosis after cardiac arrest measured from a blood of the subject.

Further, there are further provided other methods and systems to implement the present disclosure, as well as non-transitory computer-readable recording medium having stored thereon a computer program for executing the methods.

According to the present disclosure, a biological information is calculated based on a signal relating to a hemoglobin concentration measured from a cerebral region of a subject to be measured and information about prognosis after cardiac arrest of the subject is provided with reference to the calculated biological information and a biomarker relating to the prognosis after cardiac arrest measured from a blood of the subject. This makes it possible to accurately predict neurological prognosis of the patient resuscitated after cardiac arrest.

Further, according to the present disclosure, biological information and a biomarker that are not affected (or slightly affected) by a sedative drug, a neuromuscular blocker or the like used when applying a targeted temperature management to the patient resuscitated after cardiac arrest in an acute stage, are utilized. This makes it possible to accurately predict neurological prognosis during the targeted temperature management or during administration of a drug to the patient.

Furthermore, according to the present disclosure, comparing to a method of predicting neurological prognosis after cardiac arrest in the prior art, a good neurological prognosis as well as a poor neurological prognosis may be predicted. This makes it possible to establish a more accurate and appropriate treatment and management plan for the patient resuscitated after cardiac arrest.

### BRIEF DESCRIPTION OF THE DRAWINGS

FIG. 1A illustratively shows an external configuration of a prognosis information provision system according to one embodiment of the present disclosure.

FIG. 1B illustratively shows an external configuration of a prognosis information provision system according to one embodiment of the present disclosure.

FIG. 1C illustratively shows an external configuration of a prognosis information provision system according to one embodiment of the present disclosure.

FIG. 1D illustratively shows an external configuration of a prognosis information provision system according to one embodiment of the present disclosure.

FIG. 2 illustratively shows an internal configuration of the prognosis information provision system according to one embodiment of the present disclosure.

FIG. 3 illustratively shows information about results obtained by analyzing a wavelet phase coherence (WPCO) by a frequency range with respect to subjects to be measured according to one embodiment of the present disclosure.

FIG. 4 illustratively shows information about results obtained by analyzing a wavelet phase coherence (WPCO) by a frequency range with respect to subjects to be measured according to one embodiment of the present disclosure.

FIG. 5 illustratively shows a result of evaluating a performance in a prognosis information provision method according to one embodiment of the present disclosure.

FIG. 6 illustratively shows a relationship between a neuron-specific enolase (NSE) concentration, a wavelet phase coherence in a frequency range of Interval III (0.05 to 0.15 Hz) and neurologic prognosis of a subject to be measured according to one embodiment of the present disclosure.

### DETAILED DESCRIPTION

In the following detailed description of the present disclosure, references are made to the accompanying drawings that show, by way of illustration, specific embodiments in which the present disclosure may be practiced. These embodiments are described in sufficient detail to enable those skilled in the art to practice the present disclosure. It is to be understood that the various embodiments of the present disclosure, although different from each other, are not necessarily mutually exclusive. For example, specific shapes, structures and characteristics described herein may be implemented as modified from one embodiment to another without departing from the spirit and scope of the present disclosure. Furthermore, it shall be understood that the positions or arrangements of individual elements within each of the disclosed embodiments may also be modified without departing from the spirit and scope of the present disclosure. Therefore, the following detailed description is not to be taken in a limiting sense, and the scope of the present disclosure, if properly described, is limited only by the appended claims together with all equivalents thereof. In the drawings, like reference numerals refer to the same or similar functions throughout the several views.

Hereinafter, preferred embodiments of the present disclosure will be described in detail with reference to the accompanying drawings to enable those skilled in the art to easily implement the present disclosure.

In the present disclosure, a signal relating to a concentration of hemoglobin to be measured in a device and a prognosis information provision system may include a signal relating to a concentration of oxy hemoglobin (HbB₂), a signal relating to a concentration of deoxy hemoglobin, and the like.

Configuration of Prognosis Information Provision System

A device 100 and internal configurations of a prognosis information provision system 200 which perform major functions to implement the present disclosure and functions of constituent elements thereof will be described below.

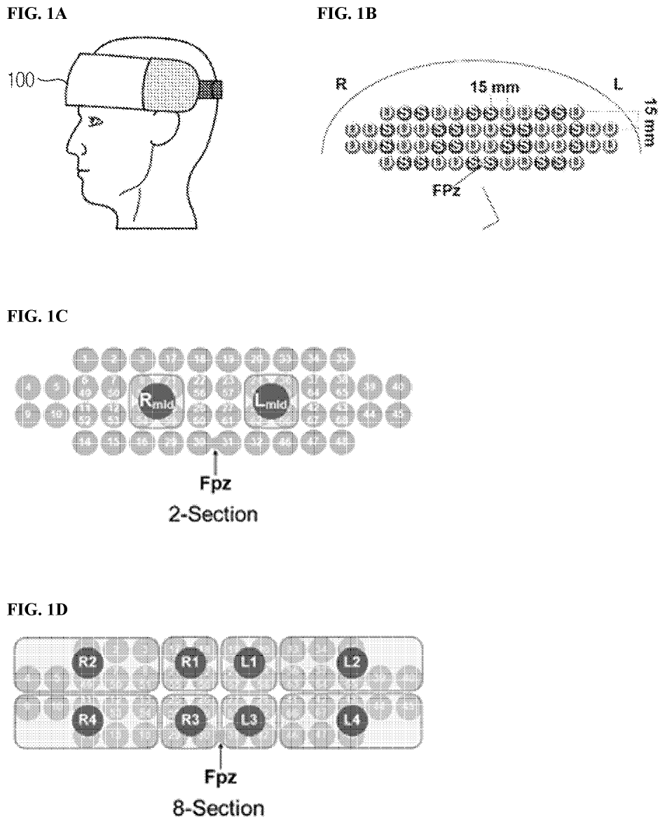

FIGS. 1A to 1D illustratively show an external configuration of a prognosis information provision system according to one embodiment of the present disclosure.

Referring to FIG. 1A, a device 100 according to one embodiment of the present disclosure may be worn on a body portion of a subject to be measured (e.g., a head portion or the like), and may perform a function of measuring a signal relating to a concentration of hemoglobin from the subject.

Specifically, the device 100 according to one embodiment of the present disclosure may include at least one light irradiation unit and at least one light detection unit, which are configured to perform a function of irradiating the head portion (more specifically, the cerebral region) of the subject with a near-infrared ray and sensing the near-infrared ray reflected or scattered from the head portion of the subject (see FIGS. 1B to 1D). For example, the signal measured by the at least one light irradiation unit and the at least one light detection unit included in the device 100 according to one embodiment of the present disclosure may be an optical density (OD) signal based on the near-infrared spectroscopy.

For example, referring to FIG. 1B, the device 100 according to one embodiment of the present disclosure may include at least one light irradiation unit and at least one light detection unit which are arranged at 15 mm intervals. In FIG. 1B, S (source) represents the light irradiation unit and D (detector) represents the light detection unit. Further, the device 100 may be worn on the head portion of the subject such that a center of the light irradiation unit or the light detection unit, or a set of the light irradiation unit and the light detection unit, which are located at the lowest position in the device 100, correspond to a frontal pole zero (Fpz) position in a 10-20 electrode arrangement (10-20 electroencephalography (EEG) system).

Further, referring to FIGS. 1C and 1D, in the device 100 according to one embodiment of the present disclosure, a predetermined number of channels may be set. Each channel may be configured with at least one light irradiation unit and at least one light detection unit. The device 100 according to one embodiment of the present disclosure may measure a signal relating to the hemoglobin concentration of the subject in such a channel.

According to one embodiment of the present disclosure, the signal relating to the hemoglobin concentration of the subject, which is measured by the device 100 according to one embodiment of the present disclosure, may include a change in concentration of oxy hemoglobin (ΔHbO₂) over time. Such a change may be calculated based on a modified Beer-Lambert's Law (MBLL). Furthermore, the signal may be filtered by taking into account at least one of a signal-to-noise ratio (SNR) and a frequency range.

The device 100 according to one embodiment of the present disclosure may measure, based on near-infrared rays respectively sensed from at least two sections included in the cerebral region of the subject, a signal relating to hemoglobin concentration for each of the at least two sections.

Specifically, according to one embodiment of the present disclosure, the cerebral region of the subject may be divided into at least two sections. Each of the at least two sections may include at least one channel. In addition, the device 100 according to one embodiment of the present disclosure may sense near-infrared rays from each of the at least two sections, and measure a signal relating to hemoglobin concentration for each of the at least two sections based on the sensed near-infrared rays.

For example, as shown in FIG. 1C, sections from which near-infrared rays reflected or scattered from the cerebral region of the subject are sensed may be divided into two sections (R_{mid} and L_{mid}). As shown in FIG. 1D, sections from which near-infrared rays reflected or scattered from the cerebral region of the subject are sensed may be divided into eight sections (R1 to R4 and L1 to L4). By dividing the sections from which the near-infrared rays reflected or scattered from the cerebral region of the subject are sensed depending on a situation, it is possible to apply an optimal measurement manner to a respective subject in consideration of a physical condition (e.g., an area or the forehead, shape of the forehead, or the like) that may be different for each subject.

The signal relating to the hemoglobin concentration measured for each of the at least two sections may mean an average of measurement values in two or more channels included in the respective section.

However, it is to be understood that the embodiments according to the present disclosure are not necessarily limited to those shown with reference to FIGS. 1A to 1D, but may be variously modified as long as it may achieve the objects of the present disclosure.

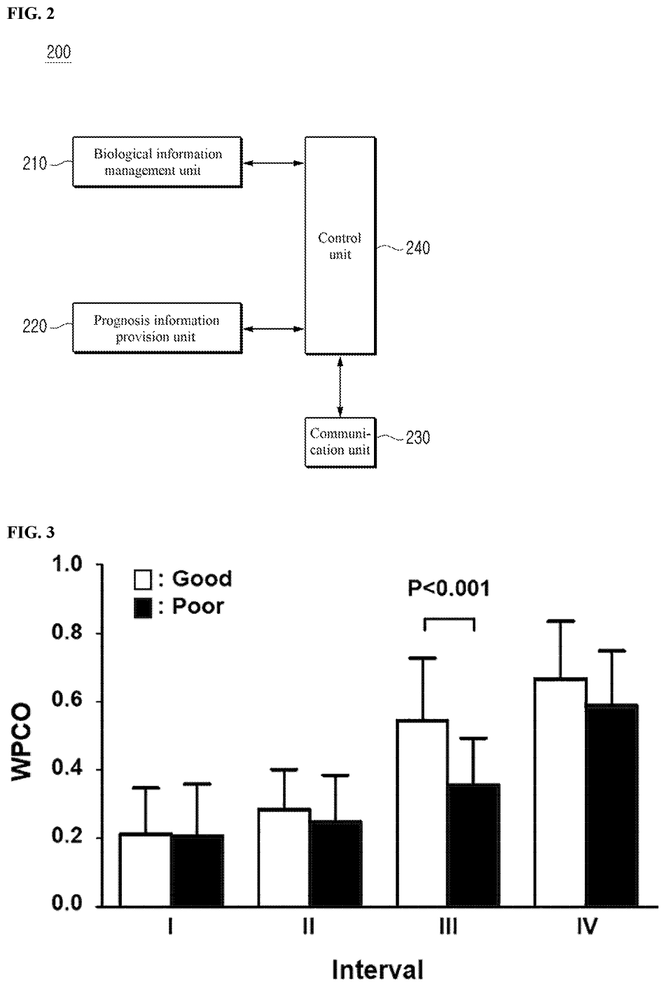

FIG. 2 illustratively shows an internal configuration of the prognosis information provision system according to one embodiment of the present disclosure.

Referring to FIG. 2, the prognosis information provision system 200 according to one embodiment of the present disclosure may be configured to include a biological information management unit 210, a prognosis information provision unit 220, a communication unit 230, and a control unit 240. According to one embodiment of the present disclosure, at least some of the biological information management unit 210, the prognosis information provision unit 220, the communication unit 230, and the control unit 240 may be program modules to communicate with an external system. Such program modules may be included in the prognosis information provision system 200 in the form of operating systems, application program modules, and other program modules, while they may be physically stored in a variety of known storage devices. Further, the program modules may also be stored in a remote storage device that may communicate with the prognosis information provision system 200. Meanwhile, the program modules may include, but not limited to, routines, subroutines, programs, objects, components, data structures, and the like for performing specific tasks or executing specific abstract data types as will be described below according to the present disclosure.

Although the prognosis information provision system 200 has been described as above, such a description is an example, and it will be apparent to those skilled in the art that at least a part of the constituent elements or functions of the prognosis information provision system 200 may be implemented inside or included in the device 100, which is a portable device worn on the body portion of the subject to be measured, as necessary.

First, the biological information management unit 210 according to one embodiment of the present disclosure may perform a function of managing the device 100 such that the at least one light irradiation unit and the at least one light detection unit included in the device 100 according to one embodiment of the present disclosure irradiate near-infrared rays to a body portion of the subject (e.g., the head portion or the like), and sense the near-infrared rays reflected or scattered from the body portion of the subject. Furthermore, the biological information management unit 210 according to one embodiment of the present disclosure may manage other functions or constituent elements of the device 100, which are necessary to measure a signal relating to hemoglobin concentration from the cerebral region of the subject.

Further, the biological information management unit 210 according to one embodiment of the present disclosure may perform a function of calculating biological information based on a signal relating to the hemoglobin concentration measured from the cerebral region of the subject.

Specifically, when the signal relating to the hemoglobin concentration from the cerebral region of the subject to be measured is measured by the device 100 according to one embodiment of the present disclosure, the biological information management unit 210 according to the embodiment of the present disclosure may calculate the biological information of the subject based on the measured signal.

More specifically, the biological information management unit 210 according to one embodiment of the present disclosure may calculate the biological information of the subject by performing a phase coherence analysis on a pair of two signals selected from a plurality of signals relating to the hemoglobin concentration measured from the cerebral region of the subject. Here, the phase coherence analysis according to one embodiment of the present disclosure may mean a series of processes that analyzes a degree of coincidence between phases of the two signals to calculate and analyze a phase coherence value. Thus, the higher the degree of coincidence between the phases of the two signals, the higher the calculated phase coherence value.

For instance, the phase coherence analysis according to one embodiment of the present disclosure may include a wavelet phase coherence (WPCO) analysis. In the WPCO analysis, the closer a WPCO value is to 1, the higher the degree of coincidence between the phases of the two signals, and the closer the WPCO value is to 0, the lower the degree of coincidence between the phases of the two signals.

Further, for example, a WPCO value of any two signals may be calculated by performing a wavelet transform on each of the two signals. Referring to Equation 1, the wavelet transform may be performed on each of two signals relating to a hemoglobin concentration of a subject to be measured, which varies with time, and subsequently, a piece of phase information about each of the two signals may be calculated. Thereafter, referring to Equation 2, a difference between the two pieces of calculated phase information may be calculated and a difference between phases of the two signals. Subsequently, referring to Equation 3, an average of the total time length of the signal with respect to each of a sine component and a cosine component of the calculated phase difference may be calculated, and the WPCO value for the two signals may be calculated by applying the calculated averages to Equation 3.

x₁(tₙ), x₂(tₙ)→φ₁(fₖ, tₙ), φ₂(fₖ, tₙ) <Equation 1>

Δφ(fₖ, tₙ)=φ₁(fₖ, tₙ)−φ₂(fₖ, tₙ) <Equation 2>

WPCO(fₖ)=\sqrt{[cos Δφ(fₖ)]²+[sin Δφ(hd k)]²} <Equation 3>

In the foregoing, it is to be understood that although the biological information calculated by the biological information management unit 210 according to one embodiment of the present disclosure is mainly described with reference to Equations 1 to 3, the present disclosure is not necessarily limited to thereto. The present disclosure may be variously modified as long as it may achieve the objects of the present disclosure.

The biological information calculated by the biological information management unit 210 according to one embodiment of the present disclosure may be calculated separately for each frequency range. The biological information separately for each frequency range may mean an average of pieces of biological information in the respective frequency range.

For example, the biological information calculated by the biological information management unit 210 according to one embodiment of the present disclosure may be assumed to have a WPCO value. In this case, the WPCO value may be calculated separately over five frequency ranges as follows.

I: 0.6 to 2 Hz (Cardiac activity)

II: 0.15 to 0.6 Hz (Respiration)

III: 0.05 to 0.15 Hz (Myogenic activity)

IV: 0.02 to 0.05 Hz (Neurogenic activity)

V: 0.0095 to 0.02 Hz (Endothelial metabolic activity)

Further, the above-described five frequency ranges may be classified depending on particular physiological origins.

The prognosis information provision unit 220 according to one embodiment of the present disclosure may perform a function of providing the information about prognosis after cardiac arrest of the subject with reference to the biological information calculated by the biological information management unit 210 according to one embodiment of the present disclosure, and a biomarker relating to prognosis after cardiac arrest, which is measured from a blood of the subject.

Specifically, based on a prognosis value derived from a prediction model having a predetermined correlation with the biological information calculated by the biological information management unit 210 according to one embodiment of the present disclosure and the biomarker relating to the prognosis after cardiac arrest, which is measured from the blood of the subject, the prognosis information provision unit 220 according to one embodiment of the present disclosure may determine the information about prognosis after cardiac arrest of the subject. Further, the prognosis information provision unit 220 according to one embodiment of the present disclosure may compare the above-described prognosis value with a preset reference value with each other to determine the information about prognosis after cardiac arrest of the subject.

For example, the biological information according to one embodiment of the present disclosure may be assumed to be a phase coherence value, and the biomarker according to one embodiment of the present disclosure may be assumed to be a concentration of neuron-specific enolase (NSE). In this case, the prediction model according to one embodiment of the present disclosure may be one having a positive correlation with the phase coherence value and having a negative correlation with the concentration of NSE. Further, the prediction model according to one embodiment of the present disclosure may utilize a logistic equation such as Equation 4. In Equation 4, NSE may represent the concentration of NSE, and Interval III may represent the WPCO value calculated in the frequency range of 0.05 to 0.15 Hz.

Further, for example, the prognosis information provision unit 220 according to one embodiment of the present disclosure may determine that, when the prognosis value derived from Equation 4 is no less than 0.2449, the prognosis after cardiac arrest of the subject is good, and when the prognosis value derived from Equation 4 is less than 0.2449, the prognosis after cardiac arrest of the subject is poor. Here, the preset reference value (e.g., 0.2449) compared with the prognosis value according to one embodiment of the present disclosure may mean the value of that the sum of sensitivity and specificity is maximal in a receiver operating characteristic (ROC) curve (i.e., an optimal cutoff value).

$${P}_{r} ( Y = good ) = \frac{1}{\begin{matrix}1 + exp ( 3.0473 + 0.0 \; 198 ⨯ \\ NSE - 8.7873 ⨯ Interval III )\end{matrix}} \qquad < Equation 4 >$$

In the foregoing, it is to be understood that although the prognosis information provision method according to one embodiment of the present disclosure is mainly described with reference to Equation 4, the present disclosure is not necessarily limited to thereto. The present disclosure may be variously modified as long as it may achieve the objects of the present disclosure.

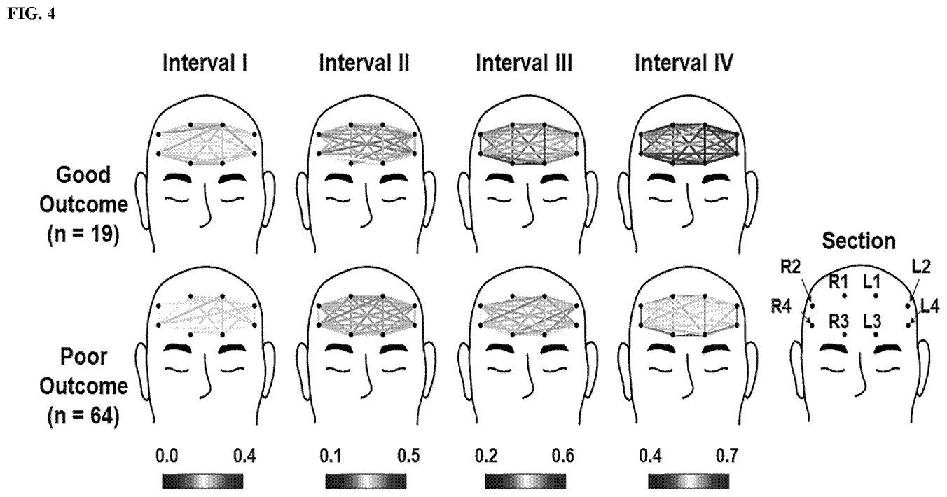

FIGS. 3 and 4 illustratively show information relating to results obtained by analyzing the wavelet phase coherence (WPCO) for each frequency range with respect to subjects according to one embodiment of the present disclosure. Herein, Interval I, II, III and IV may represent a frequency range of 0.6 to 2 Hz (cardiac activity), a frequency range of 0.15 to 0.6 Hz (respiration), a frequency range of 0.05 to 0.15 Hz (myogenic activity), and a frequency range of 0.02 to 0.05 Hz (neurogenic activity), respectively.

FIG. 3 illustrates information relating to results obtained by analyzing the WPCO based on a near infrared ray sensed respectively from two sections included in the cerebral region of the subjects, and FIG. 4 illustrates information relating to results obtained by analyzing the WPCO based on near infrared rays sensed respectively from eight sections included in the cerebral region of the subjects. In any cases, it can be seen that there is a significant difference in WPCO values between a good outcome group and a poor outcome group in terms of a neurological consequence in the frequency range of the Interval III.

The good outcome group and the poor outcome group in terms of the neurological consequence may be defined by analyzing a neurologic result of the subjects after three months from cardiac arrest and deriving a cerebral performance category (CPC) score. Specifically, the good outcome group may be defined when the derived CPC score is in a range of 1 to 2, and the poor outcome group may be defined when the derived CPC score is in a range of 3 to 5.

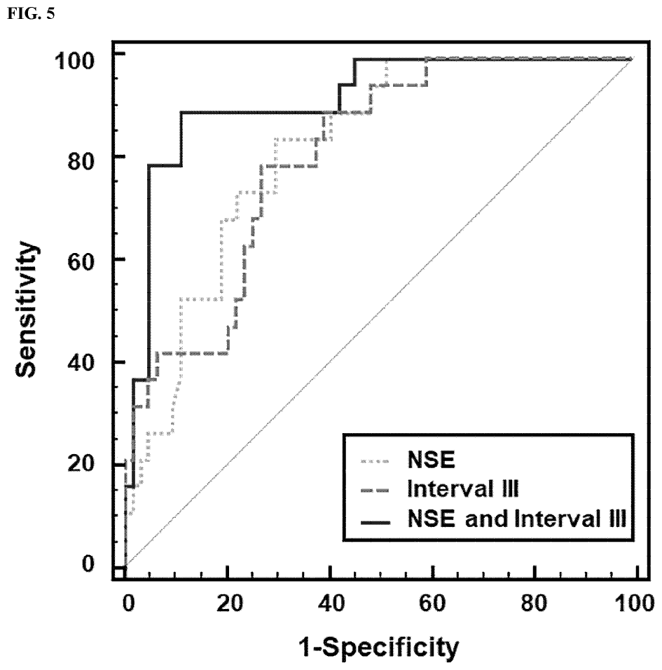

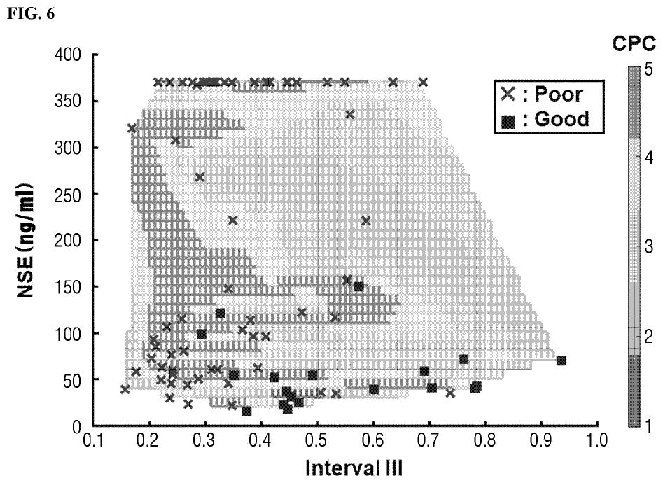

FIG. 5 illustratively shows a result of evaluating a performance in the prognosis information provision method according to one embodiment of the present disclosure.

Referring to FIG. 5, an area under curve (AUC) in the ROC curve, which represents the result of evaluating the performance of the method of predicting prognosis after cardiac arrest based on the concentration of NSE, is 0.821, and the AUC in the ROC curve, which represents the result of evaluating the performance of the method of predicting prognosis after cardiac arrest based on the WPCO value in the frequency range of Interval III, is 0.808. In contrast, according to one embodiment of the present disclosure, the AUC, which represents the result of evaluating the performance of the method of predicting prognosis after cardiac arrest based on the concentration of NSE and the WPCO value in the frequency range of Interval III, is 0.919. This shows that the evaluation method according to the present disclosure has good accuracy.

However, it is to be understood that a specific configuration relating to the prognosis information provision method according to the present disclosure is not necessarily limited to the embodiments described with reference to FIGS. 3 to 5, but may be variously modified as long as it may achieve the objects of the present disclosure.

The communication unit 230 according to one embodiment of the present disclosure may perform a function of enabling transmission/reception of data to/from the biological information management unit 210 and the prognosis information provision unit 220.

The control unit 240 according to one embodiment of the present disclosure may function to control the flow of data among the biological information management unit 210, the prognosis information provision unit 220, and the communication unit 230. That is, the control unit 240 according to the present disclosure may control the flow of data from/to the outside of the prognosis information provision system 200, or the flow of data between the constituent elements of the prognosis information provision system 200, such that the biological information management unit 210, the prognosis information provision unit 220, and the communication unit 230 may carry out their particular functions, respectively.

The embodiments according to the present disclosure as described above may be implemented in the form of program commands that can be executed by various computer components, and may be recorded on a computer-readable recording medium. The computer-readable recording medium may include program commands, data files, and data structures, independently or in combination. The program commands recorded on the computer-readable recording medium may be specially designed and configured for the present disclosure, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler, but also high-level language codes that may be executed by a computer using an interpreter. The above hardware devices may be changed to one or more software modules to perform the processes of the present disclosure, and vice versa.

Although the present disclosure has been described above in terms of specific items such as detailed constituent elements as well as the limited embodiments and the drawings, they are merely provided to help more general understanding of the present disclosure, and the present disclosure is not limited to the above embodiments. It will be appreciated by those skilled in the art to which the present disclosure pertains that various modifications and changes may be made from the above description.

Therefore, the spirit of the present disclosure shall not be limited to the above-described embodiments, and the entire scope of the appended claims and their equivalents will fall within the scope and spirit of the present disclosure.

What is claimed is:

1. A method of providing information about prognosis after cardiac arrest, the method comprising the steps of:
calculating biological information based on a signal relating to a hemoglobin concentration measured from a cerebral region of a subject to be measured; and
providing the information about the prognosis after cardiac arrest of the subject with reference to the calculated biological information and a biomarker relating to the prognosis after cardiac arrest measured from a blood of the subject.

2. The method of claim 1, wherein the signal relating to the hemoglobin concentration is measured based on a near-infrared ray sensed from the cerebral region of the subject using a near-infrared spectroscopy (NIRS).

3. The method of claim 1, wherein the biomarker comprises a concentration of a neuron-specific enolase (NSE).

4. The method of claim 2, wherein the signal relating to the hemoglobin concentration is measured for each of at least two sections included in the cerebral region of the subject based on a near-infrared ray sensed from each of the at least two sections, and
wherein the biological information is calculated based on a phase coherence analysis performed on a pair of two signals selected from measured signals relating to the hemoglobin concentration.

5. The method of claim 4, wherein the measured signals relating to the hemoglobin concentrations comprise a frequency range relating to a myogenic activity.

6. The method of claim 4, wherein the phase coherence analysis comprises a wavelet phase coherence (WPCO) analysis.

7. The method of claim 4, wherein the biomarker comprises a concentration of a neuron-specific enolase (NSE), and
wherein in the step of providing the information about the prognosis after cardiac arrest of the subject, the information about the prognosis after cardiac arrest of the subject is determined based on a prognosis value derived from a prediction model which has a positive correlation with a result of the phase coherence analysis and has a negative correlation with the concentration of the neuron-specific enolase.

8. The method of claim 7, wherein in the step of providing the information about the prognosis after cardiac arrest of the subject, the information about the prognosis after cardiac arrest of the subject is determined based on a result obtained by comparing the derived prognosis value and a preset reference value with each other.

9. A non-transitory computer-readable recording medium having stored thereon a computer program for executing the method of claim 1.

10. A system for providing information about prognosis after cardiac arrest, comprising:
a biological information management unit configured to calculate biological information based on a signal relating to a hemoglobin concentration measured from a cerebral region of a subject to be measured; and
a prognosis information provision unit configured to provide the information about the prognosis after cardiac arrest of the subject with reference to the calculated biological information and a biomarker relating to the prognosis after cardiac arrest measured from a blood of the subject.
